(19) United States
(12) Patent Application Publication
(10) Pub. No.: US 2005/0152855 A1
(43) Pub. Date: Jul. 14, 2005

(54) Materials for enhanced delivery of hydrophilic active agents in personal care formulations

(76) Inventors: Damian Hajduk, San Jose, CA (US); Marcelo Piotti, San Jose, CA (US); Miroslav Petro, San Jose, CA (US); Gerrit Klaerner, San Jose, CA (US); Adam Safir, Berkeley, CA (US); Didier Benoit, San Jose, CA (US)

Correspondence Address: DORSEY & WHITNEY LLP, IP DEPARTMENT, ATTN: SYMYX TECHNOLOGIES, 50 SOUTH SIXTH STREET, SUITE 1500, MINNEAPOLIS, MN, 55402-1498, US

(21) Appl. No.: 10/945,273

(22) Filed: Sep. 20, 2004

Related U.S. Application Data
(60) Provisional application No. 60/504,048, filed on Sep. 19, 2003.
(60) Provisional application No. 60/581,404, filed on Jun. 21, 2004.

(51) Int. Cl. A61K 7/20

(52) U.S. Cl. 424/53

(57) ABSTRACT

The present invention pertains to a phase transitional surface whitening composition. The phase transitional surface whitening composition includes a polymeric composition and an oxidizing agent. The polymeric composition of the whitening composition includes a hydrophobic component, a hydrophilic component, and an oxidizing agent harboring component. In particular, the phase transitional surface whitening composition is physiotropic.

### CROSS-REFERENCE TO RELATED APPLICATIONS

This application claims benefit under 35 U.S.C. § 119(e) to U.S. Application Ser. No. 60/504,048, filed Sep. 19, 2003, entitled “Polymers for Enhanced Teeth Whitening” (Attorney Docket No. 33315/US) and U.S. Application Ser. No. 60/581,404, filed Jun. 21, 2004, entitled “Materials for Enhanced Delivery of Hydrophilic Active Agents in Personal Care Formulations” (Attorney Docket No. 34488/US), the contents of which are incorporated herein in their entirety for all purposes.

### BACKGROUND OF THE INVENTION

Clean white healthy teeth are considered to be desirable by many people in Western countries. Dull, stained, discolored, or unhealthy looking teeth are socially objectionable to many people, both on the basis of cosmetic appearance and also as an indication of poor oral hygiene.

A tooth is comprised of an inner matrix and an outer layer of hard enamel. The enamel is the protective layer of the tooth. Generally, the enamel layer of a tooth has an opaque white or slightly off-white color. It is the enamel layer that becomes stained or discolored. The enamel layer of a tooth is composed of hydroxyapatite mineral crystals that form a partially porous surface. It is believed that the porous nature of the enamel layer is what permits staining agents and/or discoloring substances to permeate into the enamel, causing discoloration of the tooth. Many substances that an individual comes in contact with on a daily basis can.“stain” or diminish the “whiteness” of the individual's teeth. Various foods such as fruits, tomatoes, tobacco products and fluids such as wine, tea and coffee tend to stain the teeth. These produce substances that can accumulate on the enamel layer of the tooth and form a pellicle film over the teeth. These staining and discoloring substances can then permeate the enamel layer causing the teeth to have a dull or discolored appearance. This problem occurs gradually over many years, but imparts a noticeable discoloration of the enamel of an individual's teeth and reduces the “whiteness”.

Teeth whitening systems have been available since the late 1980's, acquired either through a dental professional or as an over-the-counter product. Prior to these systems, an individual desiring whiter or brighter teeth was subjected to various heat or light activated systems provided by their dental practitioner in the dental office. These practices generally involved the practitioner applying a hydrogen peroxide solution on the teeth. The sensitive soft tissues were protected with a ligated rubber dam and heat was applied to the solution to cause oxidation. Such oxidation generally removed discoloration from the tooth surfaces. These in-office procedures were generally expensive, often costing hundreds of dollars per application.

The interest in whiter teeth has created a demand for suitable affordable home use whitening products. The desirable qualities of such products include the aspects of a more convenient time and place, and/or with less equipment. Thus, numerous products have since been developed which provide an individual with the means to treat and whiten the individual's teeth in the privacy and convenience of the home. Currently, there are generally three home application methods for whitening an individual's teeth; a first method utilizes “trays”, the second method is simply painted onto the teeth, and a third is through the use of a toothpaste having an oxidizing agent, e.g. a peroxide.

In the first method, the tray provides that the whitening composition is retained at the desired location(s) and maintains contact between the tooth surface and the whitening composition. The individual typically wears the dental tray loaded with whitening composition for some extended period of time (e.g. 30 minutes to 8 hours), depending on the degree of discoloration the user desires to remove. This treatment is repeated over a sufficient period of time to effect the tooth whitening and bleaching process. Disadvantages of this type of application include that the oxidation agent in the whitening composition doesn't effectively reach the dental surface and/or is quickly dissipated.

In the second method, the tooth whitening composition is simply applied to the tooth via a brush. Generally, the individual is required to allow the coating to “dry” for at least 30 seconds and often several minutes. This can cause discomfort to the individual as the facial muscles are contracted for an extended period of time. Another drawback of this application is that the composition is generally removed by saliva in a relatively short period of time, often within a few minutes, not permitting the composition to effectively bleach and whiten the teeth.

The third method involves the incorporation of an oxidizing agent, a peroxide in a toothpaste. This is generally accomplished by toothpaste that has two main components; a first component that cleanses the teeth and a second component that is mixed with the first component, having the oxidizing agent. Generally the oxidizing agent is hydrogen peroxide. This third method has the deficiency that the duration of application of the whitening agent with the teeth is for only a short period of time; the time of brushing.

Therefore, there is a need in the art for tooth whitening compositions that overcome one or more of the afore-mentioned disadvantages of current products. Advantageously, the improved tooth whitening compositions should be user friendly and should be available at a reasonable cost.

### BRIEF SUMMARY OF THE INVENTION

The present invention, in one embodiment, provides a phase transitional surface whitening composition, i.e., dental surface whitening. The phase transitional surface whitening composition includes a polymeric composition and an oxidizing agent. The polymeric composition of the whitening composition includes a hydrophobic component, and a charged component, which may serve as an oxidizing agent harboring component. Optionally, the polymeric composition can further include a third hydrophilic component. Advantageously, the phase transitional surface whitening composition is physiotropic. The polymeric compositions of the present invention can, for example, also be useful in the delivery of biocides.

In one aspect, the physiotropic property of the phase transitional composition is based on a change in alkalinity or acidity of the environment about the phase transitional composition. In one embodiment, upon application of the phase transitional composition as a solution to the surface, an interaction with saliva occurs such that the viscosity of the phase transitional composition increases. In another aspect, the phase transitional composition is in a viscous state when applied to the surface. In particular the phase transitional composition (either formed from a solution or as a viscous material), upon application to the surface, can form an outer skin. The outer skin advantageously helps to protect the composition from being removed from the surface, and helps to provide a concentrating effect of the oxidizing agent at the appropriate site.

In certain embodiments, application of the whitening composition results in a localized viscosity increase at the interface of the environment and the composition. In particular, a skin then forms about the remaining whitening composition, thereby helping to maintain placement of the whitening composition at the desired site, as well as helping to concentrate the oxidative action of the peroxide within the composition.

Additionally, upon application of the whitening composition to a surface, there can be an increase in viscosity in the bulk state of the composition. Again, this increase in viscosity, plus the formation of an outer skin is advantageous to help maintain placement of the whitening composition at the desired site as well as concentrating the peroxide. Of course, both localized as well as bulk viscosity changes are within the scope of the present invention.

In another aspect, the polymeric resin includes an alkylacrylate, an alkylmethacrylate, or both, a (vinylbenzyl)trialkylammonium alkyl sulfonate, such as (vinylbenzyl)trimethylammonium methyl sulfonate (VBTMAMS) and, optionally, a hydroxyalkylacrylate, a hydroxyalkylmethacrylate, or both.

In one aspect, the alkylacrylate is 2-ethylhexylacrylate (EHA) and the optional hydroxyalkylacrylate is 2-hydroxyethylacrylate (HEA). In a particular embodiment, the weight % of monomers are between about 1 and about 45 weight % (vinylbenzyl)trialkylammonium alkylsulfonate, i.e., VBTMAMS, between about 0 and about 60% HEA, with the remainder being EHA, based on a total of 100 weight % of total monomer. In a particular embodiment, the weight % (vinylbenzyl)trialkylammonium alkylsulfonate, such as VBTMAMS, is between about 5 and about 15 weight %, between about 5 and about 30 weight % HEA and between about 60 and about 80 weight % EHA.

In another aspect, the alkylacrylate is 2-ethylhexylacrylate (EHA) and the optional hydroxyalkylacrylate is 2-hydroxyethylacrylate (HEA). In a particular embodiment, the weight % of monomers are between about 1 and about 39 weight % (vinylbenzyl)trialkylammonium alkylsulfonate, i.e., VBTMAMS, between about 0 and about 60% HEA, with the remainder being EHA, based on a total of 100 weight % of total monomer.

In another aspect, the weight percentage ratios of the monomers of the polymeric resin are between about 70 and about 99 weight % alkylacrylate or alkylmethacrylate or both, between about 1 and about 30 weight % (vinylbenzyl)trialkylammonium alkylsulfonate and 0 weight % of the hydroxyalkylacrylate or hydroxyalkylmethacrylate or both, based on a total of 100 weight % of total monomer.

In still another aspect, the weight percentage ratios of the monomers of the polymeric resin are between about 77 and about 90 weight % alkylacrylate or alkylmethacrylate, between about 10 and about 23 weight % (vinylbenzyl)trialkylammonium alkylsulfonate and 0 weight % of the hydroxyalkylacrylate or hydroxyalkylmethacrylate or both based on a total of 100 weight % of total monomer.

In yet another aspect, the weight percentage ratios of the monomers of the polymeric resin are between about 55 and about 99 weight % alkylacrylate or alkylmethacrylate or both, between about I and about 45 weight % (vinylbenzyl)trialkylammonium alkylsulfonate and 0 weight % of the hydroxyalkylacrylate or hydroxyalkylmethacrylate or both, based on a total of 100 weight % of total monomer.

Typically, the molecular weight (Mw) of the polymeric resin is between about 65,000 and about 300,000. Typically, the polymer of the polymeric composition is random. One method to prepare the random copolymer is by a protocol that is a radical polymerization process, e.g., a semicontinuous radical polymerization process. The polymer can be a copolymer or a terpolymer, having more than two monomeric components. Additionally, the polymer can be a block polymer. Alternatively, the polymeric composition can be a blend of polymers having the appropriate physical morphology such that the blend provides a composition useful- for whitening surfaces.

The present invention, in another embodiment, pertains to methods to treat various surfaces, including the whitening of dental surfaces. The methods include application of a composition of the invention to a suitable surface as described throughout the present specification. Advantageously, the composition of the present invention remains adhered to the surface for a period of time, sufficient to bleach or whiten the contacted surface. Generally, this can be accomplished in a few minutes, or about 30 minutes to about 2 hours. Additionally, it is believed that the compositions of the invention provide a mechanism by which the oxidizing agent is released from the composition to the appropriate surface. The compositions of the invention are unlike many products currently in the marketplace that either release the oxidizing agent too quickly such that bleaching or whitening is minimized or don't release the oxidizing agent in sufficient amounts to diminish the discoloration of, for example, the dental surface.

In yet another embodiment, the present invention provides a packaged composition as described throughout the present specification along with instructions for application of the composition to a selected surface.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the detailed description is to be regarded as illustrative in nature and not restrictive.

### DETAILED DESCRIPTION

The present invention provides unique surface whitening compositions that adhere well to various surfaces, such as dental surfaces, while still retaining the ability to release an oxidizing agent to the surface. The compositions of the invention are useful for various applications, such as hair bleaching, skin lightening, topical antiseptic application, fabric bleaching, stain treatment, as a grout cleaner, bone cleaning (fossils), oxidation of metallic surfaces and in liquid bandages.

The present invention also provides unique phase transitional surface whitening compositions. The phase transitional surface whitening compositions of the invention include a polymeric composition and an oxidizing agent. The polymeric composition of the whitening composition includes a hydrophobic component and a charged component that can be considered an oxidizing agent harboring component. Optionally, the polymeric composition can further include a third hydrophilic component. In particular, the phase transitional surface whitening composition is physiotropic.

The term “phase transitional” is intended to mean that the surface whitening compositions of the invention are sensitive to changes in the environment (physiotropicity). That is, environmental stimuli, such as a change in temperature, e.g., heating or cooling the composition (thermotropicity), a change in pH (alkalinity or acidity) (acidtropicity), subjecting the composition to shear forces (thixotropy or shear instablilty), manipulation of solvent or solvent combinations (lyotropicity), dilution of the composition (lyotropicity, coacervation), interaction with non-biological components of the surrounding environment, e.g., moisture, air, evaporation of a component of the composition, etc. can cause the composition to change from a free flowing liquid to from a viscous liquid or a viscous liquid to a free flowing liquid.

The phrase “surface whitening composition” is intended to mean that combination of the polymers described throughout the present specification, in combination with an oxidizing agent and, optionally, a carrier.

The phrase “dental surface” is intended to include those dental surfaces recognized in the art, including but not limited to, teeth, dentures, crowns and implants. In application of the present invention, the dental surface can be dry or, more particularly, can be moistened with saliva or water.

The phrases “polymer” or “polymeric composition” are recognized in the art and are intended to include polymeric resins that are copolymers, random or otherwise, and blends of polymers or copolymers. In one aspect of the invention, the polymeric composition includes a hydrophobic component and at least one charged component that can be considered the oxidizing agent harboring component, as further defined herein. Optionally, the polymeric composition can further include a hydrophilic component as well.

The phrase “random polymeric resin” is recognized in the art and is intended to include polymeric resins that are random co- or terpolymers. In one aspect of the invention, the polymeric resin includes EHA, HEA and a (vinylbenzyl)trialkylammonium alkyl sulfonate, such as VBTMAMS. In another aspect of the invention, the polymeric resin is a random co or terpolymer prepared by a free radical process, e.g., a semicontinuous free radical process.

Graft polymers and block polymers, including but not limited to linear diblocks and linear triblocks, are included within the scope of the invention and can be prepared by suitable methods known in the art.

Polymerization of the monomers can be conducted according to conventional methods, such as bulk polymerization or by semi-continuous polymerization. For example, the polymeric resin can be obtained by dissolving requisite monomers in an appropriate solvent, then conducting a polymerization reaction in the presence of a free radical initiator, such as an azo compound.

Organic solvents suitable for polymerization reactions of the invention include, for example, ketones, ethers, polar aprotic solvents, esters, aromatic solvents and aliphatic hydrocarbons, both linear and cyclic. Exemplary ketones include methyl ethyl ketone (2-butanone) (MEK), acetone and the like. Exemplary ethers include alkoxyalkyl ethers, such as methoxy methyl ether or ethyl ether, tetrahydrofuran, 1,4 dioxane and the like. Polar aprotic solvents include dimethyl formamide, dimethyl sulfoxide and the like. Polar protic solvents include water, alcohols and the like. Suitable esters include alkyl acetates, such as ethyl acetate, methyl acetate and the like. Aromatic solvents include alkylaryl solvents, such as toluene, xylene and the like and halogenated aromatics such as chlorobenzene and the like. Hydrocarbon type solvents include, for example, hexane, cyclohexane and the like.

The polymerization conditions that can be used include temperatures for polymerization typically in the range of from about 20° C. to about 110° C., more specifically in the range of from about 50° C. to about 90° C. and even more specifically in the range of from about 60° C. to about 80° C. The atmosphere can be controlled, with an inert atmosphere being advantageous, such as nitrogen or argon. In certain instances, the molecular weight of the polymer can be controlled via adjusting the ratio of monomers and free radical initiator.

A free radical initiator is provided in the polymerization mixture, which provides free radical generation upon heating or light activation. In the latter case the initiator is added to the polymerization mixture at a concentration high enough for an acceptable polymerization rate.

The phrase “free-radical initiator,” within the context of the invention, refers broadly to any and all compounds or mixtures of compounds that can lead to the formation of radical species under appropriate working conditions (thermal activation, irradiation, redox conditions, etc.).

Polymerization conditions also include the time for reaction, which can be from about 0.5 hours to about 72 hours, and more particularly in the range of from about 1 hour to about 24 hours, and even more particularly in the range of from about 2 hours to about 12 hours. Conversion of monomer to polymer is at least about 50%, more particularly at least about 75% and even more particularly at least about 90% or greater.

The initiators employed in the present invention can be a commercially available free-radical initiator. In general, however, initiators having a short half-life at the polymerization temperature are utilized in particular. More specifically, suitable free radical initiators include any thermal, redox or photo initiators, including, for example, alkyl peroxides, substituted alkyl peroxides, aryl peroxides, substituted aryl peroxides, acyl peroxides, alkyl hydroperoxides, substituted alkyl hydroperoxides, aryl hydroperoxides, substituted aryl hydroperoxides, heteroalkyl peroxides, substituted heteroalkyl peroxides, heteroalkyl hydroperoxides, substituted heteroalkyl hydroperoxides, heteroaryl peroxides, substituted heteroaryl peroxides, heteroaryl hydroperoxides, substituted heteroaryl hydroperoxides, alkyl peresters, substituted alkyl peresters, aryl peresters, substituted aryl peresters, azo compounds and halide compounds. Specific initiators include cumene hydroperoxide (CHP), t-butyl hydroperoxide (TBHP), t-butyl perbenzoate (TBPB), sodium carbonateperoxide, benzoyl peroxide (BPO), lauroyl peroxide (LPO), methylethylketone peroxide 45%, potassium persulfate, ammonium persulfate, 2,2-azobis(2,4-dimethyl-valeronitrile) (VAZO®-65), 1,1-azobis(cyclo-hexanecarbonitrile) (VAZO®-40), 2,2-azobis(N,N′-dimethyleneisobutyramidine) dihydrochloride (VAZO®-044), 2,2-azobis(2-amidino-propane) dihydrochloride (VAZO®-50) and 2,2-azobis(2-amido-propane) dihydrochloride. Redox pairs such as persulfate/sulfite and Fe(2⁺)/peroxide are also useful. Initiation may also be by heat or UV light, as is known in the art, depending on the embodiment being practiced. Those of skill in the art can select a proper initiator within the scope of this invention.

In another aspect of the invention, the polymeric composition is a random copolymer prepared by a living free radical process, e.g., a semicontinuous free radical process.

Chain transfer agents (CTAs) are known in the art and are used to help control free radical polymerizations. Ultimately, many different types of CTAs can be incorporated into the terminus of a polymer. Examples of suitable CTAs useful in the present invention include those described in U.S. Pat. No. 6,512,021, WO98/01478, WO99/35177, WO99/31144, WO99/05099 and WO98/58974, each of which is incorporated herein by reference.

Additional examples include CTAs described in U.S. Pat. Nos. 6,395,850, 6,518,364, U.S. patent application Ser. No. 10/407,405, entitled “Cleaving and Replacing Thio Control Agent Moieties from Polymers made by Living-Type Free Radical Polymerization” filed on Apr. 3, 2003 (attorney docket number 2000-089CIP3) and U.S. patent application Ser. No. 10/104,740, filed Mar. 22, 2002, the teachings of which are incorporated herein by reference in their entirety.

The use and mechanism of reversible control agents for free radical polymerization is now generally known and coined as RAFT (Reversible Addition Fragmentation Transfer), see for example, U.S. Pat. No. 6,153,705, WO 98/01478, WO 99/35177, WO 99/31144, and WO 98/58974, each of which is incorporated herein by reference. Recently new agents have been disclosed which are readily available for polymerizing desired monomers under commercially acceptable conditions, which include high conversion at the shortest possible reaction times and lower temperatures, see for example U.S. Pat. Nos. 6,380,335, 6,395,850, and 6,518,364, each of which is incorporated herein by reference.

Use of CTAs helps to control the polydispersity of the polymer. Polydispersity or the polydispersity index (PDI) of a polymer are recognized in the art and refer to the ratio of the weight average molecular weight to the number average molecular weight. In one aspect of the invention, the polydispersity (PDI) of the polymeric resins of the invention can be between about 1.5 to about 2.5, in particular, below about 2.0, more particularly between about 1.2 and about 1.5, and more specifically not more than about 1.4.

In still another aspect, the polymeric composition can be a blend of polymers, such as a hydrophobic, optionally hydrophilic, cationic and/or anionic polymer. In still yet another aspect, the polymeric composition can be a blend of copolymers, such as a hydrophobic, hydrophilic, and cationic polymer with a hydrophobic, hydrophilic and anionic copolymer. Alternatively, the polymeric composition can be a blend of copolymers, such as hydrophobic with cationic components with hydrophilic and anionic components.

The term “hydrophobic component” is recognized in the art and is intended to include those monomers, generally acrylates, methacrylates, allyl esters and vinyl monomers, that include a hydrophobic moiety, e.g., esters having branched or unbranched, substituted or unsubstituted alkyl or cycloalkyl chains of generally more than two carbon atoms. Suitable examples include, but are not limited to, substituted and unsubstituted styrenes, vinyl acetate and esters of acrylic acid and methacrylic acid, where the ester group has a carbon atom chain of at least two carbon atoms, such as ethyl acrylate or methacrylate, propyl acrylate or methacrylate, butyl acrylate or methacrylate and C12 through C18 esters of acrylic acid or methacrylic acid.

Hydrophobic components also include allyl esters, wherein the alkyl portion is a branched or unbranched, substituted or unsubstituted alkyl chain of 2 or more carbon atoms or a substituted or unsubstituted aryl group. Dialkyl and monoalkyl acrylamides and methacrylamides are further contemplated as hydrophobic components. Dialkyl methacrylamides and acrylamides include alkyl chains having at least 2 or more carbon atoms that can be substituted or unsubstituted, branched or unbranched. Monoalkyl methacrylamide or acrylamides include an alkyl chain having at least 4 carbon atoms or more that can be branched or unbranched, substituted or unsubstituted.

Surprisingly it has been found that the polymers of the invention can contain 50% or more hydrophobic component(s) by weight percentage of the polymer while retaining appropriate properties for applications. In particular, certain embodiments have polymeric components that have between about 60% to about 98% by weight percent hydrophobic component(s), more particularly 70% to about 90% by weight percent hydrophobic component(s) and even more particularly, between about 50% and 75% percent by weight hydrophobic component(s) of the total weight of the polymer. Not to be limited by theory, it is believed that the hydrophobic component(s) of the polymer provide self-association aspects to the rheological properties of the polymers of the invention. It is also believed that the hydrophobic component of the polymer creates a barrier to diffusion of the hydrophilic oxidizing agent(s) in the formulation.

The term “hydrophilic component” is recognized in the art and is intended to include those monomers, generally acrylates, methacrylates and vinyl monomers, that include a hydrophilic moiety, e.g., alkoxyesters having alkyl or cycloalkyl chains having one or more hydroxyl, polyethoxy, polypropyloxy groups attached thereto. Suitable examples include, but are not limited to N-vinyl pyrrolidone, hydroxyethyl acrylate or methacrylate, dimethylamino acrylate or methacrylate, N-tris(hydroxymethyl)acrylamide or methacrylamide, N-acryloyl or methacryloyl morpholine, “PEGylated” or “ethoxylated” acrylates or methacrylates, such as 2-(2-ethoxyethoxy)ethyl acrylate or methacrylate, N-isopropyl acrylamide or methacrylamide, glycerol mono acrylate or methacrylate, N-(2-hydroxypropyl) acrylamide or methacrylamide and N-(2-hydroxyethyl) acrylamide and methacrylamide.

The phrase “oxidizing agent harboring component” is intended to include cationic and anionic monomers. Once polymerized into a polymer or a copolymer, these moieties serve to associate with the oxidizing agent and help to stabilize the agent until applied to a surface. The association can be via charge interactions or other physical interactions.

The phrase “charged component” is recognized in the art and is intended to include those hydrophilic monomers that contain a cationic or anionic charge associated with the molecule. Such monomers, cationic or anionic, have anions or cations associated with them and are well recognized in the art. For example, the anions can include an ammonium ion, quaternary ammonium ions, halides, sulfate, carbonate, phosphate, phosphite, acetate and the like. Suitable cations include various metal ions such as sodium, potassium, calcium, etc.

Suitable cationic monomers include, but are not limited to, for example, vinyl imidazole, dimethylaminopropyl acrylate or methacrylate, N-[2-(acryloyloxy)alkylenel]trialkylammonium salts, e.g., [2-(acryloyloxy)ethyl]trimethylammonium chloride, dimethylaminoethyl acrylate or methacrylate and salts thereof, quaternary vinylbenzyl trialkylammonium salts, such as vinylbenzyltrimethylammonium chloride, quaternary allyl trialkylammonium salt, 2-aminoethyl (meth)acrylate hydrochloride, quaternary (meth)acrylamidoalkyl trialkylammonium salts, such as N-[2-(meth)acrylamido)ethyl]trimethyl ammonium chloride, quaternary vinyloxycarbonylalkyl trialkylammonium salts, such as (vinyloxycarbonylmethyl)trimethylammonium chloride, and quaternary allyloxycarbonylalkyl trialkylammonium salts such as allyloxycarbonylmethyl)trimethylammonium chloride, and dimethyl diallyl ammonium chloride (DMDAAC).

Suitable anionic monomers include, but are not limited to, acrylic acid, mono-2-(meth)acryloyl)ethyl succinate, ethylene glycol (meth)acrylate phosphate, 2-(meth)acrylamido-2-methyl-1-propanesulfonic acid and vinyl phosphoric acid.

The polymeric compositions of the invention generally include between about 60% and about 95% (weight percent) of a hydrophobic component and between about 40% and about 5% (weight percent) of a cationic component and, optionally, hydrophilic component. In certain embodiments, the weight percent of the hydrophobic component of the polymeric composition is between about 65% and about 90%, in particular between about 70% and about 85% and more specifically between about 75% and about 80%. Consequently, the weight percent of the cationic/hydrophilic component of the polymeric composition is between about 35% and about 10%, in particular between about 30% and about 15% and more specifically between about 25% and about 20%.

Alternatively, the polymeric composition includes at least about 50% (weight percent) of a hydrophilic component with the remainder (50% weight percent) being an anionic component and, optionally, a hydrophobic component. In certain embodiments, the weight percent of the hydrophilic component of the polymeric composition is between about 55% and about 95%, in particular between about 65% and about 85% and more specifically between about 75% and about 80%. Consequently, the weight percent of the anionic/hydrophilic component of the polymeric composition is between about 45% and about 5%, in particular between about 35% and about 15% and more specifically between about 25% and about 20%.

In yet another aspect, the polymeric composition of the invention is a blend of the hydrophobic/cationic polymer (optionally with a hydrophilic component) and the hydrophilic/anionic polymer (optionally with a hydrophobic component).

Not to be limited by theory, it is believed that the charged component helps to stabilize the oxidizing agent and possibly complexes with the oxidizing agent. It is also believed that the charged monomer helps to solubilize the overall hydrophobic polymer in an aqueous delivery vehicle. The charged component of the polymer may precipitate from the aqueous solution upon contact with saliva or biological fluids in a phenomenon known as coacervation. Additionally, it is believed that the charged component of the polymer helps to adhere/adsorb the composition onto the surface. Where anionic components are contained within the polymer, it is believed that the anionic portion helps to selectively deliver the composition to the surface, thereby helping to protect the gums.

In certain embodiments, the polymeric resin of the composition includes an alkylacrylate, an alkylmethacrylate, or both, such as 2-ethylhexyl acrylate (EHA), optionally, a hydroxyalkylacrylate, a hydroxyalkylmethacrylate, or both, such as 2-hydroxyethylacrylate (HEA) and a (vinylbenzyl)trialkylammonium alkylsulfonate (VBTAAAS), such as (vinylbenzyl)trimethylammonium methyl sulfonate (VBTMAMS).

It should be understood by those skilled in the art that the terms “alkane sulfonate” and “alkylsulfonate” are intended to include alkanes as well as alkane radicals. For example, methane sulfonate and methyl sulfonate denote the same compound and the terms can be used interchangeably.

In one embodiment, the formulations of the invention contain polymers that wet surfaces such as hydroxyapatite. The formulations of the invention retain cohesive strength in the presence of multiple washes with aqueous solutions, such as with artificial saliva or saliva. The formulations also have a rate at which they release peroxide over time. The latter was measured in order to describe the relationship between polymer composition and performance.

The exploration of a range of monomers of the present invention allowed identification of polymers that exhibit a combination of good cohesive strength, good adhesion to a surface, e.g., teeth and a controlled release of hydrogen peroxide. Without being limited by theory, it is believed that the polymers of the invention contain sufficient cationic groups to induce a transition to a high viscosity state (or even a gel) upon exposure to an aqueous environment, such as the inside of the mouth. This gel resists dissolution or disintegration upon exposure to the aqueous environment, e.g., saliva, or to mechanical perturbation such as that produced by the natural rubbing process of the internal tissues of the mouth. The polymer also keeps a delicate hydrophilicity/hydrophobicity balance. It is believed that polymers that are too hydrophobic present low adhesion to the surface (teeth) or are even insoluble in water/ethanol mixtures. It is also believed that a very hydrophilic material will release the hydrogen peroxide too quickly or disintegrate immediately upon contact with an aqueous treatment, such as saliva.

The combinations of the properties of the polymers of the invention provide an improvement over existing commercial surface whiteners, e.g., tooth whiteners. In general, some commercially available products that are hydrophilic in nature, tend to disintegrate within a few minutes upon contact with an, aqueous based solution, such as saliva, resulting in a very short hydrogen peroxide/surface (teeth) contact time. Other commercial whiteners which are hydrophobic in nature generally show good cohesive strength, but they generally do not adhere well to many surfaces, such as wet teeth, and they tend to release hydrogen peroxide extremely slowly.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-prop an-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 15 carbon atoms (C₁-C₁₅ alkyl), more preferably from 1 to 10 carbon atoms (C₁-C₁₀ alkyl) and even more preferably from 1 to 6 carbon atoms (C₁-C₆ alkyl or lower alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl,” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Cycloalkyl,” by itself or as part of another substituent, refers to a saturated or unsaturated cyclic alkyl radical, as defined herein. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. Preferably, the cycloalkyl group comprises from 3 to 10 ring atoms (C₃-C₁₀ cycloalkyl) and more preferably from 3 to 7 ring atoms (C₃-C₇ cycloalkyl).

The term “alkylacrylate” or “alkylmethacrylate” is recognized in the art and is intended to include the reaction product of, for example, acrylic acid or acryoyl chloride and a suitable alcohol to form an ester of acrylic acid or methacrylic acid. Suitable examples include methyl acrylate and methacrylate, ethyl acrylate and methacrylate, 2-ethylhexyl acrylate and methacrylate, etc.

The term “hydroxyalkylacrylate” or “hydroxyalkylmethacrylate” is recognized in the art and is an ester of acrylic acid or methacrylic acid having a hydroxyl moiety on the alkyl portion of the ester chain. Suitable examples include 2-hydroxyethylacrylate and methacrylate, 3-hydroxypropylacrylate and methacrylate, and 2-hydroxypropylacrylate and methacrylate, etc.

The term “(vinylbenzyl)trialkylammonium alkylsulfonate” refers to a monomer that is the reaction product of (vinylbenzyl)-N,N-dialkylamine and an alkane alkylsulfonate, e.g., methyl alkylsulfonate, or methyl methanesulfonate (R¹, R², R³ and R⁴ each are methyl), as shown in Scheme 1.

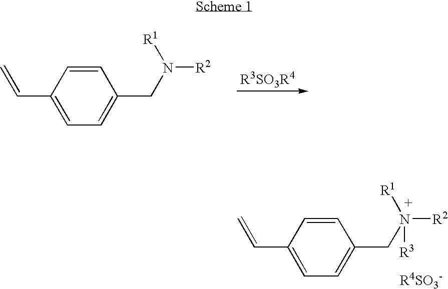

It should be understood by one skilled in the art that preparation of the vinylbenzyl)trialkylammonium alkylsulfonate can include vinyl isomers at the 4, 3 and 2 positions, due to manufacturing processes. As depicted, the 4-position isomer is representative.

It should also be understood that R¹, R², R³ and R⁴, each independently, are alkyl groups having a carbon atom chain from about 1 carbon atom to about 15 carbon atoms in length. In certain embodiments, R¹ and R² are the same and are selected from the group of methyl, ethyl, propyl, butyl, pentyl, hexyl, heptyl, and the alkyl groups can be branched or unbranched. R³ and R⁴ are often different and are each independently selected from the group of methyl, ethyl, propyl, butyl, pentyl, hexyl, heptyl, and the alkyl groups can be branched or unbranched.

In certain embodiments, R¹ and R² are the same branched or unbranched alkyl groups and are selected from the group of methyl, ethyl, propyl, butyl, pentyl, hexyl and heptyl, R³ is branched or unbranched and is selected from the group of methyl, ethyl, propyl, butyl, pentyl, hexyl and heptyl, and R⁴ is branched or unbranched and selected from the group of methyl, ethyl, propyl, butyl, pentyl, hexyl and heptyl.

In a particular embodiment, the weight % of monomers are between about 1 and about 45 weight % (vinylbenzyl)trialkylammonium alkylsulfonate, i.e., VBTMAMS, between about 0 and about 60% of hydroxyalkylacrylate, hydroxyalkymethacrylate, or both (e.g., HEA), with the remainder being an alkylacrylate, alkylmethacrylate, or both (e.g., EHA), based on a total of 100 weight % of total monomer.

In another embodiment, the weight % of monomers are between about 1 and about 45 weight % (vinylbenzyl)trialkylammonium alkylsulfonate, i.e., VBTMAMS, between about 0 and about 60% of alkylacrylate, alkylmethacrylate, or both (e.g., EHA), with the remainder being a hydroxyalkylacrylate, hydroxyalkylmethyacrylate, or both (e.g., HEA), based on a total of 100 weight % of total monomer.

In a particular embodiment, the weight % of (vinylbenzyl)trialkylammonium alkylsulfonate, such as VBTMAMS, is between about 5 and about 15, the weight % of a hydroxyalkylacrylate, hydroxyalkylmethacrylate, or both (e.g., HEA) is between about 5 and about 30 and the weight % of an alkylacrylate, alkylmethacrylate, or both (e.g., EHA) is between about 55 and about 90.

In a another embodiment, the weight % of (vinylbenzyl)trialkylammonium alkylsulfonate, such as VBTMAMS, is between about 9 and about 12, the weight % of a hydroxyalkylacrylate, a hydroxyalkylmethacrylate, or both (e.g., HEA) is between about 5 and about 25 and the weight % of an alkylacrylate, alkylmethacrylate, or both (e.g., EHA) is between about 53 and about 86.

In another specific embodiment, the weight % of (vinylbenzyl)trialkylammonium alkylsulfonate, such as VBTMAMS, is between about 9 and about 12, the weight % of a hydroxyalkylacrylate, a hydroxyalkylmethacrylate, or both (e.g., HEA) is between about 10 and about 20 and the weight % of an alkylacrylate, alkylmethacrylate or both (e.g., EHA) is between about 68 and about 81.

In still another specific embodiment, the weight % of (vinylbenzyl)trialkylammonium alkylsulfonate, such as VBTMAMS, is between about 9 and about 12, the weight % of a hydroxyalkylacrylate, hydroxyalkylmethacrylate, or both (e.g., HEA) is between about 5 and about 10 and the weight % of an alkylacrylate, alkylmethacrylate, or both (e.g., EHA) is between about 78 and about 86.

In one aspect, the weight % of (vinylbenzyl)trialkylammonium alkylsulfonate, such as VBTMAMS, is between about 5 and about 15 and the weight % of a hydroxyalkylacrylate, hydroxyalkylmethacrylate, or both (e.g., HEA) is between about 5 and about 30, with the remainder being an alkylacrylate, alkylmethacrylate, or both (e.g., EHA), based on a total of 100 weight % of total monomer.

In another aspect, the weight % of (vinylbenzyl)trialkylammonium alkylsulfonate, such as VBTMAMS, is between about 9 and about 12 and the weight % of a hydroxyalkylacrylate, hydroxyalkylmethacrylate, or both (e.g., HEA) is between about 5 and about 25, with the remainder being an alkylacrylate, alkylmethacrylate, or both (e.g., EHA), based on a total of 100 weight % of total monomer.

In still another aspect, the weight % of (vinylbenzyl)trialkylammonium alkylsulfonate, such as VBTMAMS, is between about 9 and about 12 and the weight % of a hydroxyalkylacrylate, hydroxyalkylmethacrylate, or both (e.g., HEA) is between about 10 and about 20, with the remainder being an alkylacrylate, alkylmethacrylate, or both (e.g., EHA), based on a total of 100 weight % of total monomer.

In still yet another aspect, the weight % of (vinylbenzyl)trialkylammonium alkylsulfonate, such as VBTMAMS, is between about 9 and about 12 and the weight % of a hydroxyalkylacrylate, hydroxyalkylmethacrylate, or both (e.g., HEA) is between about 5 and about 10, with the remainder being an alkylacrylate, alkylmethacrylate, or both (e.g., EHA), based on a total of 100 weight % of total monomer.

The phrase “oxidizing agent” is recognized in the art and is intended to include bleaching agents and peroxygens, such as peroxides. Suitable peroxides include, for example, hydrogen peroxide, urea peroxide, calcium peroxide, pyrophosphate peroxidate, carbamide peroxide, sodium carbonate peroxide, enzyme oxidases, peroxymonosulfuric acid and its salts, monoperoxysulfate, oxopiperidinium methosulfate, peroxyacetic acid and its salts, perboric acid and its salts, peroxydiphosphate and its salts and sodium perborate. Commercially available peroxides are available under various tradenames including, for example, Albone® (Atofina), Hioxyl® (Adams Health), Hipox, Hybrite® (FMC), Inhibine, Oxydol, Oxyfull, Oxysept, Peroxal® (Atofina), Perhydrol, Perone, Peroxaan, Superoxol, Valsterane® (Atofina).

Typically, an effective amount of oxidizing agent useful in a surface treatment, such as in a dental surface whitening composition, is between about 2% and about 25% by weight based-on the weight of the polymeric resin. In particular, the usefuil range of oxidizing agent is between about 5% and about 20% by weight and more particularly between about 5% and about 10% by weight. Generally, an effective amount of an oxidizing agent is that amount that helps to remove the discoloration of the surface.

In certain embodiments, the oxidizing agent, such as hydrogen peroxide, is commercially available as an aqueous solution (30% aqueous solution hydrogen peroxide, Aldrich Chemical Company). Therefore, the above-identified weight percentages are based on the weight of the 30% aqueous solution of hydrogen peroxide. In other embodiment, where the oxidizing agent is in solid form, then the percentage is based on the actual weight of the solid. Such determinations can be made by those skilled in the art.

The whitening composition of the present invention retains the oxidizing agent within the composition and releases it to the surface over a period of time. In one embodiment, more than 10% of the oxidizing agent remains retained in the composition after 5 minutes upon contact. In another embodiment, less than 10% of the oxidizing agent remains retained in the composition after 6 hours. In still another embodiment, more than 10% of the oxidizing agent remains retained in the composition after 3 hours. In still yet another embodiment, more than 10% of the oxidizing agent remains retained in the composition after 1 hour and in still another embodiment, more than 10% of the oxidizing agent remains retained in the composition after 30 minutes.

The phrase “carrier” is recognized in the art and is intended to include suitable aqueous and nonaqueous solvents such as water, ethanol, isopropyl alcohol, methyl ethyl ketone, polyols (such as glycerol, propylene glycol, polyethylene glycol, and the like), hydrocarbons (such as isopentane or hexane), vegetable oils, such as olive oil, and organic esters, such as ethyl oleate and suitable mixtures thereof.

Proper fluidity can be maintained, for example, by the- use of combinations of solvents such that the viscosity of the whitening composition of the invention can be attenuated as desired. In certain circumstances, the solvent choice provides that the composition is a free flowing liquid, having a low viscosity at ambient temperature (35% ethanol 65% water by. volume) or can be a viscous fluid at ambient temperature (less than 20% ethanol, remainder water by volume). In some instances, no alcohol is present in the composition.

Typically, the compositions of the invention are combined with an aqueous solution having an alcohol, ethanol for example, for delivery. The percentage of alcohol to water can range from about 10 percent to about 60 percent by volume. Typically, between about 20 to about 40 percent polymeric resin is combined with the aqueous/alcoholic solution (weight to weight) to afford the composition along with an effective amount of oxidizing agent.

In certain aspects of the invention, the molecular weight (Mw) of the polymeric resin is between about 65,000 and about 300,000, more particularly between about 75,000 and about 200,000, and more specifically about 100,000. The polymeric resin(s) of the invention essentially have an absence of low molecular weight polymeric residues having a Mw of about 50,000 or less.

In still yet another aspect of the invention, the composition has a relative ratio of EHA, HEA and a trialkylammonium salt (as described herein) such that an increase in viscosity occurs when contacted with a moist environment, such as that of saliva. Alternatively, the composition may undergo an increase in viscosity when subjected to a change in pH or temperature.

It should be understood that the term “saliva” is recognized in the art and is intended to include saliva that is produced naturally by a being, but also includes artificial saliva. Artificial saliva is a material that is reconstituted from the main components of human and animal saliva.

In one embodiment, the present invention provides compositions that are useful as surface whitening compositions, e.g., teeth whitening compositions. The unique properties of the polymeric resins are a result of the combination of the monomeric components as outlined throughout the specification. The polymers are designed to deliver the oxidizing agent, i.e., peroxide, to the surface, and at the same time, form a film that prevents the oxidizing agent from leaching out of the formulation, for example, in to the saliva environment of an oral cavity. Application of the formula permits the release of the oxidizing agent to the surface.

In one embodiment, the compositions of the invention change morphology when certain environmental stimuli act upon the composition. The compositions are generally utilized as a liquid. In this context, the term “liquid” is intended to include a solution, emulsion, dispersion, suspension or gel. Not to be limited by theory, it is believed the morphological characteristics of the composition can change and are at least partially dependent upon the solvent system among other processing factors. In certain aspects, the “liquid” may be a free flowing liquid, that once applied to a surface, becomes a viscous material.

The viscosity of the formulation can be attenuated by a skilled artisan, for example, by manipulation of the solvent system. It has been observed that application of the composition to a surface in a moist environment results in an increase in viscosity to the applied composition. This increase in viscosity occurs at least at the interface between the moist environments, e.g., saliva, and composition, and can also occur in the bulk phase or both. Again, not to be limited by theory, it is believed that contact with moisture at the surface (e.g., dental) interface helps to facilitate the increase in viscosity, at least at the local environment proximate to the dental surface. The increase in viscosity of the composition provides the advantage that the material is not easily removed and adheres well to the surface, thus helping to concentrate and/or maintain the oxidizing agent at the surface, e.g., the dental surface.

The unique properties of the compositions of the invention provide that the composition is suitable for overnight application, especially in dental applications. In one embodiment, the composition adheres to the surface for at least 30 minutes, and more particularly, at least 2 hours. In certain embodiments, the composition remains on the surface in the following relative ratios as the whitening composition wears away: at least about 100% of the composition remains after about 5 minutes; at least about 80% of the composition remains after about 30 minutes; at least about 5% of the composition remains after about 180 minutes.

The present invention overcomes several of the disadvantages of currently marketed tooth whitening compositions. For example, certain tooth whitening compositions do not adhere well to the dental surface. In this instance, the oxidizing agent is not in contact with the surface for a sufficient period of time to bleach away discoloration. Also, certain currently available tooth whitening compositions adhere to the dental surface but do not release sufficient quantities of the oxidizing agent from the composition to allow bleaching of the surface to occur.

As described throughout the present application, the whitening compositions of the present invention adhere well to surfaces, i.e., a dental surface. Not to be limited by theory, it is believed this is at least partially accomplished by the increased viscosity of the composition partially or substantially remaining in contact with the surface, for example, a dental surface and/or saliva. The resultant increase in viscosity provides that the material remains on the dental surface for an extended period of time, as described throughout the application. The retention on the surface then permits delivery of the oxidizing agent to the surface. The engineered polymeric resins of the invention release the oxidizing agent over an extended period of time, facilitating removal of discoloration of the surface.

The present invention provides methods to treat surfaces. The methods include application of the compositions of the invention, as described herein, to a surface, such as a dental surface. The whitening composition can be applied to a dry surface or a moistened surface, e.g., saliva can be present on the surface. Advantageously, the composition remains adhered to the surface for a period of time sufficient to bleach or whiten the contacted surface. Generally, this can be accomplished in minutes, or about 30 minutes to about 2 hours.

Application of the composition can be effected by coating the surface by techniques known in the art. These include, for example, by an applicator, by spraying onto the surface, or by use with a strip as is known in the art. Applicators include, for example, a brush or sponge. In certain dental applications, this can be accomplished by use of a syringe if a preformed tray is used.

The present invention also provides packaged surface whitening compositions as described throughout the present specification along with instructions for application of the surface whitening composition to a surface. Generally, the whitening composition is applied as described above for a period of at least about 30 minutes to at least about 2 hours. In a particular embodiment, the application is left on a dental surface overnight, e.g., about 8 hours.

The whitening compositions of the invention can be removed, at anytime, by brushing the surface with a suitable cleansing agent, such as water, or in a particular embodiment, toothpaste. Alternatively, the composition can be removed by simply wiping the treated surface vigorously with a dry or moist cloth.

In certain aspects regarding dental applications, the composition can be removed by use of a mouthwash (often containing a surfactant), or by slow dissolution by saliva and mechanical action during eating. Alternatively, the composition can be removed by natural abrasion in the oral cavity.

In yet another method, the composition can be removed by rinsing with mouthwash or a salt solution.

### EXAMPLE

1) Synthesis

Examples of Random Copolymerization of Different Monomers

For Examples 1 through 7 listed below, the following degassed stock solutions can be prepared:
a. 94.9 mg of AIBN in 10 ml of DMF
b. 4.17 g of (acryloyloxy)ethyl]trimethylammonium chloride (AETMAC) (80% solution in water) in 10 ml of water
c. 4.99 g of n-Hexylacrylate (HA) in 30 ml of DMF
d. 1.66 g of Vinylpyrrolidone (VPL) in 10 ml of DMF
e. 4.99 g of n-Butylacrylate (BA) in 30 ml of DMF
f. 1.66 g of N,N-Dimethylacrylamide (DMA) in 10 ml of DMF
g. 1.66 g of 2-Hydroxyethyl acrylate (HEA) in 10 ml of DMF

### Example 1

### Molar Feed Ratio: 89.88% BA, 10.12% AETMAC

In a reaction vessel can be added 2.48 ml of DMF, 0.195 ml of solution a, 0.043 ml of solution b, and 0.46 ml of solution e. The reaction can then be heated at 80° C., and while stirring are added simultaneously during 2 hours in a semi-continuous way 0.292 ml of solution a in 10 equal portions, 0.389 ml of solution b in 100 equal portions, and 4.14 ml of solution e in 100 equal portions. After the addition is complete the mixture can be kept at 80° C. for one more hour and then can be cooled down to room temperature. The mixture can be concentrated by rotatory evaporation and the residue can be redissolved in a minimum amount of ethanol. The polymer can then be precipitated in excess hexane. The oil formed can be decanted and dried under vacuum.

### Example 2

### Molar Feed Ratio: 88.07% HA, 11.93% AETMAC

In a reaction vessel can be added 2.48 ml of DMF, 0.195 ml of solution a, 0.043 ml of solution b, and 0.46 ml of solution c. The reaction can then be heated at 80° C., and while stirring are added simultaneously during 2 hours in a semi-continuous way 0.292 ml of solution a in 10 equal portions, 0.389 ml of solution b in 100 equal portions, and 4.14 ml of solution c in 100 equal portions. After the addition is complete the mixture can be kept at 80° C. for one more hour and then can be cooled down to room temperature. The mixture can be concentrated by rotatory evaporation and the residue redissolved in a minimum amount of ethanol. The polymer can then be precipitated in excess hexane. The oil formed can be decanted and dried under vacuum.

### Example 3

### Molar Feed Ratio: 80.72% BA, 10.19% DMA, 9.09% AETMAC

In a reaction vessel can be added 2.45 ml of DMF, 0.198 ml of solution a, 0.04 ml of solution b, 0.425 ml of solution e and 0.04 ml of solution f. The reaction can then be heated at 80° C., and while stirring are added simultaneously during 2 hours in a semi-continuous way 0.297 ml of solution a in 10 equal portions, 0.360 ml of solution b in 100 equal portions, 3.825 ml of solution e in 100 equal portions, 0.360 ml of solution f in 100 equal portions. After the addition is complete the mixture can be kept at 80° C. for one more hour and then can be cooled down to room temperature. The mixture can then be concentrated by rotatory evaporation and the residue can be redissolved in a minimum amount of ethanol. The polymer can then be precipitated in excess hexane. The oil formed can be decanted and dried under vacuum.

### Example 4

### Molar Feed Ratio: 77.68% HA, 11.80% DMA, 10.52% AETMAC

In a reaction vessel can be added 2.45 ml of DMF, 0.198 ml of solution a, 0.04 ml of solution b, 0.425 ml of solution c and 0.04 ml of solution f. The reaction can then be heated at 80° C., and while stirring are added simultaneously during 2 hours in a semi-continuous way 0.297 ml of solution a in 10 equal portions, 0.360 ml of solution b in 100 equal portions, 3.825 ml of solution c in 100 equal portions, 0.360 ml of solution f in 100 equal portions. After the addition is complete the mixture can be kept at 80° C. for one more hour and can then be cooled down to room temperature. The mixture can be concentrated by rotatory evaporation and the residue can be redissolved in a minimum amount of ethanol. The polymer can then be precipitated in excess hexane. The oil formed can be decanted and dried under vacuum.

### Example 5

### Molar Feed Ratio: 78.68% HA, 10.66% VPL, 10.66% AETMAC

In a reaction vessel can be added 2.45 ml of DMF, 0.198 ml of solution a, 0.04 ml of solution b, 0.425 ml of solution c and 0.04 ml of solution d. The reaction can then be heated at 80° C., and while stirring are added simultaneously during 2 hours in a semi-continuous way 0.297 ml of solution a in 10 equal portions, 0.360 ml of solution b in 100 equal portions, 3.825 ml of solution c in 100 equal portions, 0.360 ml of solution d in 100 equal portions. After the addition is complete the mixture can be kept at 80° C. for one more hour and can then be cooled down to room temperature. The mixture can be concentrated by rotatory evaporation and the residue can be redissolved in a minimum amount of ethanol. The polymer can then be precipitated in excess hexane. The oil formed can be decanted and dried under vacuum.

### Example 6

### Molar Feed Ratio: 83.02% BA, 7.63% HEA, 9.35% AETMAC

In a reaction vessel can be added 2.45 ml of DMF, 0.198 ml of solution a, 0.04 ml of solution b, 0.425 ml of solution e and 0.04 ml of solution g. The reaction can then be heated at 80° C., and while stirring are added simultaneously during 2 hours in a semi-continuous way 0.297 ml of solution a in 10 equal portions, 0.360 ml of solution b in 100 equal portions, 3.825 ml of solution e in 100 equal portions, 0.360 ml of solution g in 100 equal portions. After the addition is complete the mixture can be kept at 80° C. for one more hour and can then be cooled down to room temperature. The mixture can be concentrated by rotatory evaporation and the residue can be redissolved in a minimum amount of ethanol. The polymer can then be precipitated in excess hexane. The oil formed can be decanted and dried under vacuum.

### Example 7

### Molar Feed Ratio: 79.05% HA, 10.25% HEA, 10.71% AETMAC

In a reaction vessel can be added 2.45 ml of DMF, 0.198 ml of solution a, 0.04 ml of solution b, 0.425 ml of solution e and 0.04 ml of solution g. The reaction can then be heated at 80° C., and while stirring are added simultaneously during 2 hours in a semi-continuous way 0.297 ml of solution a in 10 equal portions, 0.360 ml of solution b in 100 equal portions, 3.825 ml of solution e in 100 equal portions, 0.360 ml of solution g in 100 equal portions. After the addition is complete the mixture can be kept at 80° C. for one more hour and can then be cooled down to room temperature. The mixture can be concentrated by rotatory evaporation and the residue can be redissolved in a minimum amount of ethanol. The polymer can then be precipitated in excess hexane. The oil formed can be decanted and dried under vacuum.

For more information regarding general synthesis utilizing a semicontinuous polymerization reactor (SCPPR), see for example, U.S. patent application Ser. No. 09/873,176, filed on Jun. 1, 2001 entitled “Parallel Semicontinuous or Continuous Reactors” (attorney docket number 1998-014), the contents of which are incorporated herein by reference in their entirety.

Synthesis of Hydrophilic, Peroxide Concentrating Polymers

### Example 8

### Acrylic Acid/N-vinylpyrrolidone Random Copolymer

In a reaction vessel were added 10 ml of degassed water, 5 ml of degassed isopropanol, 1.87 g of N-vinylpyrrolidone, 0.135 g of acrylic acid, 0.66 ml of a solution prepared with 100 mg of N,N,N′,N′-tetramethylethylenediamine in 10 ml of water, and 0.66 ml of a solution prepared with 100 mg of potassium persulphate in 10 ml of water. The reaction was then stirred at room temperature overnight. The resulting solution was dialyzed against distilled water. The polymer was then freeze/dried. The polymer had a Mw of 180000, a polydispersity of 1.4 and contained an acrylic acid/N-vinylpyrrolidone ratio of 40/60 as determined by ¹H-NMR.

Acrylic acid/N-vinylpyrrolidone polymers with acrylic acid content ranging form 5 to 80% and acrylic acid/N-vinylpyrrolidone/n-butylacrylate ternary polymers containing up to 80% content of n-butyl acrylate were prepared with similar procedures by changing the monomer feed ratio.

Preparation of Cationic Polymer/Peroxide Concentrating Polymer Blend.

### Example 9

### Hexylacrylate/N-vinylpyrrolidone/AETMAC Random Copolymer

10 ml of a 10% aqueous solution of a cationic polymer (50.66% HA, 38.68% VPL, 10.66% AETMAC) was mixed with 1 ml of a 10% aqueous solution of polymer from example 8. To the homogeneous mixture was carefully added, without stirring, a drop of a pH 7 buffer. The change of pH in the areas where the polymer solution entered in contact with the buffer induced the formation of a white skin that created a physical barrier separating the polymer solution from the buffer solution.

### Example 10

### Synthesis of (vinylbenzyl)trimethylammonium methanesulfonate

To a solution of 16.6g of N-(4-Vinylbenzyl)-N,N-dimethylamine in 100 mL of tetrahydrofuran was added slowly 11 g of methyl methanesulfonate. The reaction mixture was then stirred at room temperature for 12 h. The reaction mixture was filtered off to collect a white precipitate washed with THF. The 18.27 g of Hygroscopic solid collected were dried overnight, characterized by ¹H NMR and immediately mixed with 54.81 g of deionized water and 58.4 mg of methoxy phenol as the stock formulation ready to be used for the polymerization reactions.

Polymer Library Synthesis

A triangular library of 150 wells was designed with Library Studio® (Symyx Technologies, Inc., Santa Clara, Calif., USA). The following monomers and compositions were defined as shown in Table 1.
(vinylbenzyl)trimethylammonium methanesulfonate, ranging from 1% to 3 5% of total monomer, from bottom to top;
2-hydroxyethylacrylate, ranging from 0% to 70% of total monomer, from left to right;

2-ethylhexylacrylate, completing the monomeric composition.

TABLE 1

| Library | | mol | | Cohesion | |
|---|---|---|---|---|---|
| element | mol EHA | VBTMAMS | mol HEA | (% retained) | Adhesion |
| A1 | 0.650 | 0.350 | 0.000 | 2.68 | 4 |
| B1 | 0.674 | 0.326 | 0.000 | 2.67 | 4 |
| B2 | 0.624 | 0.326 | 0.050 | 3.74 | 4 |
| C1 | 0.699 | 0.301 | 0.000 | 4.16 | 4 |
| C2 | 0.649 | 0.301 | 0.050 | 4.96 | 4 |
| C3 | 0.599 | 0.301 | 0.100 | 4.31 | 4 |
| D1 | 0.723 | 0.277 | 0.000 | 2.06 | 4 |
| D2 | 0.673 | 0.277 | 0.050 | 3.92 | 4 |
| D3 | 0.623 | 0.277 | 0.100 | 4.07 | 4 |
| D4 | 0.573 | 0.277 | 0.150 | 2.01 | 4 |
| E1 | 0.747 | 0.253 | 0.000 | 1.28 | 4 |
| E2 | 0.697 | 0.253 | 0.050 | 1.27 | 4 |
| E3 | 0.647 | 0.253 | 0.100 | 1.56 | 4 |
| E4 | 0.597 | 0.253 | 0.150 | 1.75 | 4 |
| E5 | 0.547 | 0.253 | 0.200 | 2.14 | 4 |
| F1 | 0.771 | 0.229 | 0.000 | 0.75 | 4 |
| F2 | 0.721 | 0.229 | 0.050 | 0.98 | 4 |
| F3 | 0.671 | 0.229 | 0.100 | 2.76 | 4 |
| F4 | 0.621 | 0.229 | 0.150 | 3.27 | 4 |
| F5 | 0.571 | 0.229 | 0.200 | 3.44 | 4 |
| F6 | 0.521 | 0.229 | 0.250 | 2.60 | 4 |
| G1 | 0.796 | 0.204 | 0.000 | 1.01 | 4 |
| G2 | 0.746 | 0.204 | 0.050 | 0.89 | 4 |
| G3 | 0.696 | 0.204 | 0.100 | 1.26 | 4 |
| G4 | 0.646 | 0.204 | 0.150 | 0.98 | 4 |
| G5 | 0.596 | 0.204 | 0.200 | 1.50 | 4 |
| G6 | 0.546 | 0.204 | 0.250 | 2.49 | 4 |
| G7 | 0.496 | 0.204 | 0.300 | 2.18 | 4 |
| H1 | 0.820 | 0.180 | 0.000 | 60.87 | 3 |
| H2 | 0.770 | 0.180 | 0.050 | 1.72 | 4 |
| H3 | 0.720 | 0.180 | 0.100 | 0.30 | 4 |
| H4 | 0.670 | 0.180 | 0.150 | 0.69 | 4 |
| H5 | 0.620 | 0.180 | 0.200 | 1.91 | 4 |
| H6 | 0.570 | 0.180 | 0.250 | 2.68 | 4 |
| H7 | 0.520 | 0.180 | 0.300 | 3.88 | 4 |
| H8 | 0.470 | 0.180 | 0.350 | 3.28 | 4 |
| I1 | 0.844 | 0.156 | 0.000 | 90.94 | 2 |
| I2 | 0.794 | 0.156 | 0.050 | 20.24 | 3 |
| I3 | 0.744 | 0.156 | 0.100 | 0.41 | 3 |
| I4 | 0.694 | 0.156 | 0.150 | 2.06 | 4 |
| I5 | 0.644 | 0.156 | 0.200 | 0.79 | 4 |
| I6 | 0.594 | 0.156 | 0.250 | 3.11 | 4 |
| I7 | 0.544 | 0.156 | 0.300 | 3.20 | 4 |
| I8 | 0.494 | 0.156 | 0.350 | 3.58 | 4 |
| I9 | 0.444 | 0.156 | 0.400 | 3.19 | 4 |
| J1 | 0.869 | 0.131 | 0.000 | 103.20 | 1 |
| J2 | 0.819 | 0.131 | 0.050 | 103.62 | 1 |
| J3 | 0.769 | 0.131 | 0.100 | 91.89 | 2 |
| J4 | 0.719 | 0.131 | 0.150 | 20.07 | 3 |
| J5 | 0.669 | 0.131 | 0.200 | 0.75 | 3 |
| J6 | 0.619 | 0.131 | 0.250 | 0.50 | 4 |
| J7 | 0.569 | 0.131 | 0.300 | 0.53 | 4 |
| J8 | 0.519 | 0.131 | 0.350 | 1.26 | 4 |
| J9 | 0.469 | 0.131 | 0.400 | 2.36 | 4 |
| J10 | 0.419 | 0.131 | 0.450 | 3.92 | 4 |
| K1 | 0.893 | 0.107 | 0.000 | 117.75 | 1 |
| K2 | 0.843 | 0.107 | 0.050 | 99.54 | 1 |
| K3 | 0.793 | 0.107 | 0.100 | 95.66 | 2 |
| K4 | 0.743 | 0.107 | 0.150 | 84.44 | 2 |
| K5 | 0.693 | 0.107 | 0.200 | 95.67 | 3 |
| K6 | 0.643 | 0.107 | 0.250 | 33.33 | 3 |
| K7 | 0.593 | 0.107 | 0.300 | 1.12 | 4 |
| K8 | 0.543 | 0.107 | 0.350 | 0.67 | 4 |
| K9 | 0.493 | 0.107 | 0.400 | 2.61 | 4 |
| K10 | 0.443 | 0.107 | 0.450 | 2.90 | 4 |
| K11 | 0.393 | 0.107 | 0.500 | 4.24 | 4 |
| L1 | 0.917 | 0.083 | 0.000 | 68.65 | 1 |
| L2 | 0.867 | 0.083 | 0.050 | 58.06 | 1 |
| L3 | 0.817 | 0.083 | 0.100 | 85.96 | 1 |
| L4 | 0.767 | 0.083 | 0.150 | 104.43 | 1 |
| L5 | 0.717 | 0.083 | 0.200 | 97.45 | 1 |
| L6 | 0.667 | 0.083 | 0.250 | 93.94 | 2 |
| L7 | 0.617 | 0.083 | 0.300 | 115.62 | 4 |
| L8 | 0.567 | 0.083 | 0.350 | 54.87 | 4 |
| L9 | 0.517 | 0.083 | 0.400 | 2.19 | 4 |
| L10 | 0.467 | 0.083 | 0.450 | 1.30 | 4 |
| L11 | 0.417 | 0.083 | 0.500 | 1.95 | 4 |
| L12 | 0.367 | 0.083 | 0.550 | 3.12 | 4 |
| M1 | 0.941 | 0.059 | 0.000 | insoluble | 0 |
| M2 | 0.891 | 0.059 | 0.050 | insoluble | 0 |
| M3 | 0.841 | 0.059 | 0.100 | 93.11 | 1 |
| M4 | 0.791 | 0.059 | 0.150 | 99.09 | 1 |
| M5 | 0.741 | 0.059 | 0.200 | 108.72 | 1 |
| M6 | 0.691 | 0.059 | 0.250 | 108.47 | 1 |
| M7 | 0.641 | 0.059 | 0.300 | 109.37 | 2 |
| M8 | 0.591 | 0.059 | 0.350 | 122.88 | 3 |
| M9 | 0.541 | 0.059 | 0.400 | 109.35 | 4 |
| M10 | 0.491 | 0.059 | 0.450 | 44.87 | 4 |
| M11 | 0.441 | 0.059 | 0.500 | 9.40 | 4 |
| M12 | 0.391 | 0.059 | 0.550 | 3.72 | 4 |
| M13 | 0.341 | 0.059 | 0.600 | 4.39 | 4 |
| N1 | 0.966 | 0.034 | 0.000 | insoluble | 0 |
| N2 | 0.916 | 0.034 | 0.050 | insoluble | 0 |
| N3 | 0.866 | 0.034 | 0.100 | insoluble | 0 |
| N4 | 0.816 | 0.034 | 0.150 | insoluble | 0 |
| N5 | 0.766 | 0.034 | 0.200 | insoluble | 0 |
| N6 | 0.716 | 0.034 | 0.250 | 65.95 | 2 |
| N7 | 0.666 | 0.034 | 0.300 | 122.23 | 3 |
| N8 | 0.616 | 0.034 | 0.350 | 87.23 | 3 |
| N9 | 0.566 | 0.034 | 0.400 | 103.56 | 4 |
| N10 | 0.516 | 0.034 | 0.450 | 95.07 | 4 |
| N11 | 0.466 | 0.034 | 0.500 | 91.48 | 4 |
| N12 | 0.416 | 0.034 | 0.550 | 28.08 | 4 |
| N13 | 0.366 | 0.034 | 0.600 | 24.68 | 4 |
| N14 | 0.316 | 0.034 | 0.650 | 9.34 | 4 |
| O1 | 0.990 | 0.010 | 0.000 | insoluble | 0 |
| O2 | 0.940 | 0.010 | 0.050 | insoluble | 0 |
| O3 | 0.890 | 0.010 | 0.100 | insoluble | 0 |
| O4 | 0.840 | 0.010 | 0.150 | insoluble | 0 |
| O5 | 0.790 | 0.010 | 0.200 | insoluble | 0 |
| O6 | 0.740 | 0.010 | 0.250 | insoluble | 0 |
| O7 | 0.690 | 0.010 | 0.300 | insoluble | 0 |
| O8 | 0.640 | 0.010 | 0.350 | insoluble | 0 |
| O9 | 0.590 | 0.010 | 0.400 | insoluble | 0 |
| O10 | 0.540 | 0.010 | 0.450 | insoluble | 0 |
| O11 | 0.490 | 0.010 | 0.500 | insoluble | 0 |
| O12 | 0.440 | 0.010 | 0.550 | insoluble | 0 |
| O13 | 0.390 | 0.010 | 0.600 | insoluble | 0 |
| O14 | 0.340 | 0.010 | 0.650 | insoluble | 0 |
| O15 | 0.290 | 0.010 | 0.700 | insoluble | 0 |

Adhesion scores: (See performance experiments)
0 Polymer not soluble in formulation and therefore was not tested.
1 Poor
2, 3 Mediocre
4 Good
   Table 1 provides formulation cohesive and adhesive strength as a function of polymer composition at constant solids fraction and solvent composition.

All reactions were done on 8 mL scale, using DMF as the solvent and with a total monomeric concentration of 10 wt %. As the cationic monomer stock solution is prepared in water, a small amount of water was present in all the reactions. The amount of initiator used was 1 wt % compared to the total monomer in the mixture. The following stock solutions were used for the syntheses:
2-Ethylhexylacrylate: 15 wt % in DMF
2-Hydroxyethylacrylate: 15 wt % in DMF
(Vinylbenzyl)trimethylammonium methanesulfonate: 25 wt % in water
AIBN: 1 wt % in DMF
The addition protocol was as follows:

In a first stage, the solvent, 10% of each of the monomers, and 40% of the initiator was added. Stirring and heating of the solution was begun. Once the temperature of the reaction mixture reached 80° C., the second stage was begun. During the second stage, the rest of the monomers were added semicontinuously in 100 steps during 2 hours, together with the remaining 60% of the initiator, in this case in 10 steps during the same time. Heating was maintained for 3 hours at 80° C. The process was performed under 100 psi N₂. The reaction mixtures were transferred to glass vials and dried under vacuum using a GeneVac® evaporator. The polymers were used for the performance experiments with no further purification.

Performance Experiments

Synthesized polymers (100 mg) were combined with water (75 ul), ethanol (35 ul), and a non-interactive and non diffusive fluorescent pigment (Fluorescent tempera, a pinch) to create primary formulations. Adhesion was estimated by placing a plastic pipette tip filled with approximately 15 mg of each formulation against a glass surface and qualitatively estimating the degree to which formulation expelled from the tip adhered to the surface. Cohesion was estimated by placing ˜10 mg of each formulation in the wells of a microtiter plate, measuring the amount of light emitted at 560 nm by reflectivity upon excitation with 520 nm irradiation run on a Spectra Max® Gemini EM microplate reader system in order to determine the initial amount of formulation in each well, washing each well with artificial saliva, and determining the fraction of the original material remaining in each well as described elsewhere. The amount of material remaining after five washes with artificial saliva, expressed as a percentage of the original amount, was used as a measure of cohesion. Representative cohesion measurements, showing the fraction of selected formulations remaining in the plate after each wash with artificial saliva, appear in Table 2.

[image]

Based on these results, selected materials were synthesized in greater quantity by replicating the recipe specified for the appropriate cell of the primary synthesis design across multiple wells in the reactor. The materials selected were the ones containing EHA/HEANVBTMAMS molar ratios of 67/8/25, 72/8/20, 77/8/15 and 82/8/10 (See Table 3). Scale-ups were prepared in 6 g quantities, purified by precipitation in hexane, and dried under vacuum. Dry polymers were then formulated by mixing 350 mg of the polymer with 122 ul of ethanol, and 227 ul of a solution prepared by mixing 61.7 mg of ethylenediaminetetraacetic acid disodium salt (EDTA-Na₂, Aldrich) with 100 ml 30% H₂O₂ (Aldrich.)

Small quantities of each secondary formulation were combined with dye in order to measure the cohesive strength of the formulation in the presence of peroxide. The results were in agreement with the measurements made in the absence of peroxide on the primary formulations. Adhesive strength was measured by soaking a glass slide coated with a ceramic layer of hydroxyapatite (HA) in artificial saliva for 1 minute. The excess saliva was eliminated by holding the slide in vertical position for another minute. With the slide in horizontal position, a plastic tip impregnated with the polymer formulation was put in contact with the wet HA surface. If the gel adhered immediately to the surface, the adhesion was deemed to be “good.” If no adhesion was observed, adhesion was rated “poor.” Anything in between was considered “mediocre.” The results obtained were also in agreement with those obtained in primary screening.

Peroxide release profiles were measured by introducing 10 mg of each secondary formulation to the bottom of a 4 mL Titeseal shell vial. 2×250 mL of artificial saliva was introduced carefully into the vial. Mixture was left on titer plate shaker to provide gentle stirring. At a given time after addition of the artificial saliva, all the saliva was taken off carefully from the comer of the bottom of the vial to avoid any gel removal. Operation was repeated for different times 25 ul of the removed saliva were dissolved into 2.5 mL of deionized water. 20 uL of the resulting solution were placed into 8 column wells of a 8×12 plastic microtiter plate. One additional column contained 20 ul of deionized water and is used as reference to subtract the background effect. To each vial were added 180 ul of a 20% KI solution in deionized water. The hydrogen peroxide released into the saliva was titrated by UV spectroscopy by measuring the absorbance at 350 nm associated with the formation of a potassium iodide-hydrogen peroxide complex. Titration was performed on a Spectra Max® Plus microplate reader system: after 15 minutes of equilibration, reading was performed at 385nm and data were obtained from software SoftMax® Pro 4.1. For each sample, the reported value of the absorbance is given by the average of eight readings less the average of eight readings of the reference.

[image]

The peroxide release data (Table 3) indicate that the time period over which peroxide release occurs can be tuned by varying the composition of the polymer in the formulation. This enables formulators to optimize the release profile for different products, such as a rapid-acting “day product” and a sustained release “night product.”

Polymer libraries 11 through 18 were synthesized as followed and screened as described herein.
AETMAC/n-butyl acrylate (from 20/80 to 0/100 mol ratio)
AETMAC (10 mol %) and 2-ethylhexylacrylate/vinylpyrrolidinone (from 40/80 to 80/40 mol ratio)
AETMAC (10 mol %) and n-butylacrylate/2-hydroxyethylacrylate (from 20/80 to 0/100 mol ratio)
AETMAC (10 mol %) and 2-ethylhexylacrylate/2-hydroxyethylacrylate (from 40/80 to 80/40 mol ratio)
VBTMAC (10 mol %) and 2-ethylhexylacrylate/vinylpyrrolidinone (from 40/80 to 80/40 mol ratio)
VBTMAC (10 mol %) and 2-ethylhexylacrylate/2-hydroxyethylacrylate (from 40/80 to 80/40 mol ratio)
VBTMAC/2-ethylhexylacrylate (from 10/90 to 70/30 mol ratio)
VBTMAC/n-butylacrylate (from 0/100 to 30/70 mol ratio)

All reactions were done on 8 mL scale, using DMF as the solvent and with a total monomeric concentration of 10 wt %. As the cationic monomer stock solution is prepared in water, a small amount of water was present in all the reactions. The amount of initiator used was 1 wt % compared to the total monomer in the mixture. The following stock solutions were used for the syntheses (note that hydrophobic monomers include n-butylacrylate and 2-ethylhexylacrylate, hydrophilic monomers include vinylpyrrolidinone and 2-hydroxyethylacrylate, and cationic monomers are AETMAC and VBTMAC):
Hydrophobic monomer: 15 wt % in DMF
Hydrophilic monomer: 15 wt % in DMF
Cationic monomer: 25 wt % in water
AIBN: 1 wt % in DMF

The addition protocol was as follows:

In a first stage, the solvent, 10% of each of the monomers, and 40% of the initiator was added. Stirring and heating of the solution was begun. Once the temperature of the reaction mixture reached 80° C., the second stage was begun. During the second stage, the rest of the monomers were added semicontinuously in 100 steps during 2 hours, together with the remaining 60% of the initiator, in this case in 10 steps during the same time. Heating was maintained for 3 hours at 80° C. The process was performed under 100 psi N₂. The reaction mixtures were transferred to glass vials and dried under vacuum using a GeneVac® evaporator. The polymers were used for the performance experiments with no further purification.

After evaporation of the solvent, each polymer was diluted two times with a 3/7 ethanol/water mixture. After homogenization of the mixture, a small amount of inorganic fluorescent pigment was added. Two different properties were monitored: a) The formulation solution properties (such as a visual observation of the viscosity, tackiness, etc,) and the gel retention properties of the formulations, measured by the gel retention test as described previously.

The visual observations are described next to the monomer composition of each polymer in the following graphs.
T=Low viscosity, somewhat tacky clear formulation
V=Clear viscous formulation
P=Paste like viscous formulation
G=gel

[image]

[image]

[image]

[image]

[image]

[image]

[image]

[image]

SWN denotes a comparative commercially available sample of SIMPLY WHITE® NIGHT by Colgate.

Data points in the graphs above having values of greater than 100% gel retention are believed to be due to the increase of opacity of the polymer after treatment with saliva. The reflectance signal increases due to the opaqueness of the polymer.

Although the present invention has been described with reference to preferred embodiments, persons skilled in the art will recognize that changes may be made in form and detail without departing from the spirit and scope of the invention.

1. A phase transitional whitening composition, comprising
a polymer comprising a hydrophobic component and a charged component; and
an oxidizing agent, wherein the phase transitional whitening composition is physiotropic.

2. The phase transitional whitening composition of claim 1, wherein the polymer further comprises a hydrophilic component.

3. The phase transitional whitening composition of claim 1, wherein the composition is acidtropic.

4. The phase transitional whitening composition of claim 1, wherein the composition is lyotropic.

5. The phase transitional whitening composition of claim 1, wherein the polymer is a polymeric blend of homopolymers.

6. The phase transitional whitening composition of claim 1, wherein the polymer comprises a random copolymer.

7. The phase transitional whitening composition of claim 6, wherein the random copolymer is prepared by a protocol that comprises a living free radical polymerization process.

8. A method for whitening surfaces, comprising the steps
applying to a surface, a phase transitional surface whitening composition, comprising
a polymer comprising a hydrophobic component and a charged component; and
an oxidizing agent, wherein the phase transitional whitening composition is physiotropic.

9. A packaged phase transitional whitening composition, comprising
a polymer comprising a hydrophobic component and a charged component;
an oxidizing agent, wherein the phase transitional whitening composition is physiotropic; and
instructions for application of the whitening composition to a surface.

10. The packaged composition of claim 9, wherein the polymer comprises is a polymeric blend of homopolymers.

11. The-packaged composition of claim 9, wherein the polymer comprises a random copolymer.

12. The packaged composition of claim 11, wherein the random copolymer is prepared by a protocol that comprises a living free radical polymerization process.

13. A composition, comprising
a polymeric resin comprising an alkylacrylate, an alkylmethacrylate, or both, a (vinylbenzyl)trialkylammonium alkylsulfonate (VBTAAAS), and between about 0 and about 60 weight % of a hydroxyalkylacrylate, a hydroxyalkylmethacrylate, or both, based on a total of 100 weight % of total monomer; and
an oxidizing agent.

14. The composition of claim 13, wherein the (vinylbenzyl)trialkylammonium alkylsulfonate (VBTAAAS) has the formula 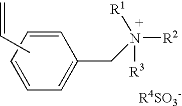
wherein R¹, R², R³ and R⁴, each independently, are alkyl groups having a carbon atom chain from about 1 carbon atom to about 15 carbon atoms in length.

15. The composition of claim 14, wherein R¹, R², R³ and R⁴, are all methyl groups.

16. The composition of claim 13, wherein the alkyl moiety of the alkylacrylate or alkylmethacrylate or both is 2-ethylhexyl, the (vinylbenzyl)trialkylammonium alkylsulfonate is (vinylbenzyl)trimethylammonium methanesulfonate, and the hydroxyalkyl moiety of the hydroxyalkylacrylate or hydroxyalkylmethacrylate or both is 2-hydroxyethyl.

17. The composition of any of claims 13-16, wherein the Mw of the polymeric resin is between about 65,000 and about 300,000.

18. A method for whitening surfaces, the method comprising the steps of:
applying to the surface, a surface whitening composition comprising
a polymeric resin comprising an alkylacrylate, an alkylmethacrylate, or both, a (vinylbenzyl)trialkylammonium alkylsulfonate (VBTAAAS), and between about 0 and about 60 weight % of a hydroxyalkylacrylate, a hydroxyalkylmethacrylate, or both, based on a total of 100 weight % of total monomer; and
an oxidizing agent.

19. The method of claim 18, wherein the (vinylbenzyl)trialkylammonium alkylsulfonate (VBTAAAS) has the formula 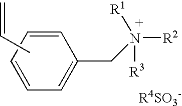
wherein R¹, R², R³ and R⁴, each independently, are alkyl groups having a carbon atom chain from about 1 carbon atom to about 15 carbon atoms in length.

20. The method of claim 19, wherein R¹, R², R³ and R⁴, are all methyl groups.

21. The method of claim 18, wherein the alkyl moiety of the alkylacrylate or alkylmethacrylate or both is 2-ethylhexyl, the (vinylbenzyl)trialkylammonium alkylsulfonate is (vinylbenzyl)trimethylammonium methanesulfonate, and the hydroxyalkyl moiety of the hydroxyalkylacrylate or hydroxyalkylmethacrylate or both is 2-hydroxyethyl.

22. The method of any of claims 18-21, wherein the Mw of the polymeric resin is between about 65,000 and about 300,000.

23. A packaged whitening composition, comprising
a polymeric resin comprising an alkylacrylate, an alkylmethacrylate or both, a (vinylbenzyl)trialkylammonium alkylsulfonate (VBTAAAS), and between about 0 and about 60 weight % of a hydroxyalkylacrylate, a hydroxyalkylmethacrylate, or both, based on a total of 100 weight % of total monomer;
an oxidizing agent; and
instructions for application of the whitening composition to a surface.

24. The packaged whitening composition of claim 23, wherein the (vinylbenzyl)trialkylammonium alkylsulfonate (VBTAAAS) has the formula 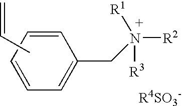
wherein R¹, R², R³ and R⁴, each independently, are alkyl groups having a carbon atom chain from about 1 carbon atom to about 15 carbon atoms in length.

25. The packaged whitening composition of claim 24, wherein R¹, R², R³ and R⁴, are all methyl groups.
